(19) United States
(12) Patent Application Publication
(10) Pub. No.: US 2011/0015752 A1
(43) Pub. Date: Jan. 20, 2011

(54) System and Method for Acetabular Cup

(75) Inventor: Jason D. Meridew, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 12/502,828

(22) Filed: Jul. 14, 2009

(51) Int. Cl. A61F 2/32; A61F 2/36

(52) U.S. Cl. 623/22.24; 623/23.11

(57) ABSTRACT

An acetabular implant assembly for replacing a portion of the anatomy is provided. The acetabular implant assembly can include a fiber-reinforced polymeric cup having an exterior surface that engages the anatomy and an interior surface. The acetabular implant assembly can also include a metal or ceramic bearing liner, which can be coupled to the interior surface of the with an acetabular implant assembly cup via a taper lock connection. The metal or ceramic bearing liner can have an interior bearing surface. The acetabular implant assembly can include a metal articulating member, which can be movable within the interior bearing surface to replace the articulating portion of the anatomy.

### INTRODUCTION

Many portions of the human anatomy naturally articulate relative to one another. Generally, the articulation between the portions of the anatomy is substantially smooth and without abrasion. This articulation is allowed by the presence of natural tissues, such as cartilage and strong bone.

Over time, however, due to injury, stress, degenerative health issues and various other issues, articulation of the various portions of the anatomy may become rough or impractical. For example, injury may cause the cartilage or the boney structure to become weak, damaged, or non-existent. Therefore, the articulation of the anatomical portions is no longer possible for the individual.

At such times, it may be desirable to replace the anatomical portions with a prosthesis such that normal or easy articulation may be reproduced. A proximal femur generally articulates within an acetabulum surface or cavity in a pelvis. After injury or other degenerative processes, the acetabulum may become rough or damaged. Therefore, it may be desirable to replace the acetabulum with a prosthesis or implant.

### SUMMARY

The present disclosure relates to biomedical implants and in particular to a system and method for an acetabular implant assembly.

In this regard, provided is an acetabular implant assembly for replacing a portion of the anatomy, which can include a fiber-reinforced polymeric cup having an exterior surface that engages the anatomy and an interior surface. The acetabular implant assembly can also include a metal or ceramic bearing liner, which can be coupled to the interior surface of the cup via a taper lock connection. The metal or ceramic bearing liner can have an interior bearing surface. The acetabular implant assembly can include an articulating member, which can be movable within the interior bearing surface to replace the articulating portion of the anatomy.

Also provided is an acetabular implant assembly for replacing an articulating portion of an anatomy. The acetabular imiant assembly can include an acetabular cup composed of a carbon fiber-reinforced polyetheretherketone material, which can have an exterior surface that engages the anatomy and an interior surface. The acetabular implant assembly can also include a metal bearing liner coupled to the interior surface of the acetabular cup via a taper lock connection. The metal bearing liner can include an articulated bearing surface. The acetabular implant assembly can include a metal femoral head implant, which can include a head that articulates within the interior surface of the bearing liner.

Further provided is a method of replacing an articulating portion of an anatomy with an acetabular implant assembly. The method can include inserting a fiber-reinforced polymeric acetabular cup into a prepared portion of the anatomy, and coupling a metal bearing liner to an interior surface of the polymeric acetabular cup. The method can also include coupling a metal femoral head implant to the metal bearing liner.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of an acetabular cup according to one of various examples;

FIG. 2 is an exploded view of the acetabular cup of FIG. 1;

FIG. 3 is a cross-sectional view of the acetabular cup of FIG. 1, taken along line 3-3 of FIG. 1;

FIG. 4 is a perspective view of an acetabular cup according to another of various examples;

FIG. 5 is an exploded view of the acetabular cup of FIG. 4;

FIG. 6 is a cross-sectional view of the acetabular cup of FIG. 4, taken along line 6-6 of FIG. 4;

FIG. 7 is an exploded view of the acetabular cup of FIG. 4 including an exemplary femoral head implant having a larger radius or diameter; and

FIG. 8 is an environmental schematic illustration of the acetabular cup of FIG. 1 coupled to the anatomy and including an exemplary femoral head implant having a larger radius or diameter.

### DETAILED DESCRIPTION

The following description is merely exemplary in nature and is not intended to limit the present disclosure, application, or uses. It should be understood that throughout the drawings, corresponding reference numerals indicate like or corresponding parts and features. As indicated above, the present teachings are directed towards providing a system and method for an acetabular cup. It should be noted, however, that the present teachings could be applicable to any appropriate implant in which it is desirable to reduce the size of the implant, such as a wall thickness of an implant, without reducing the strength of the implant. Therefore, it will be understood that the following discussions are not intended to limit the scope of the appended claims.

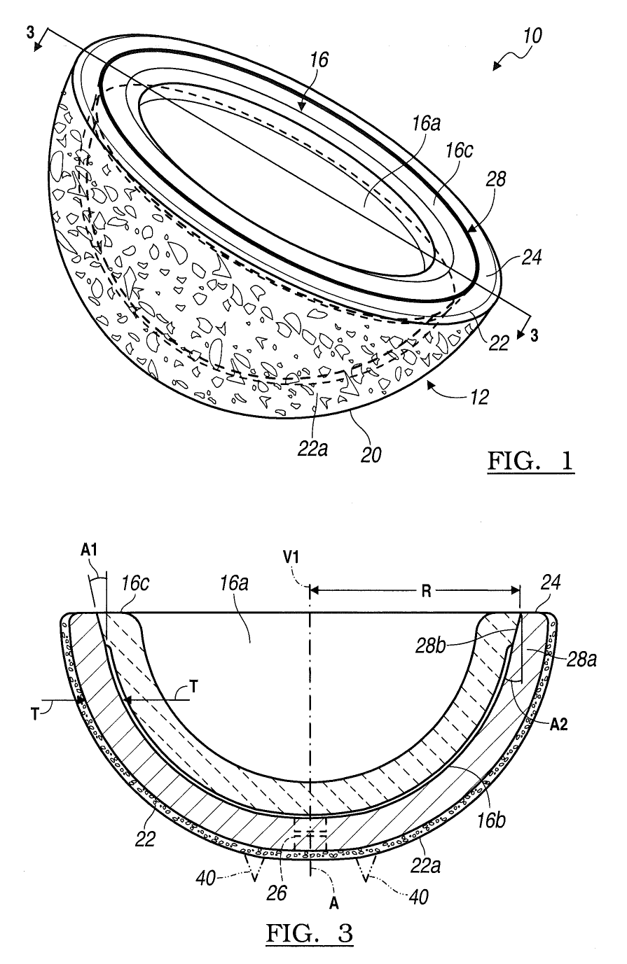

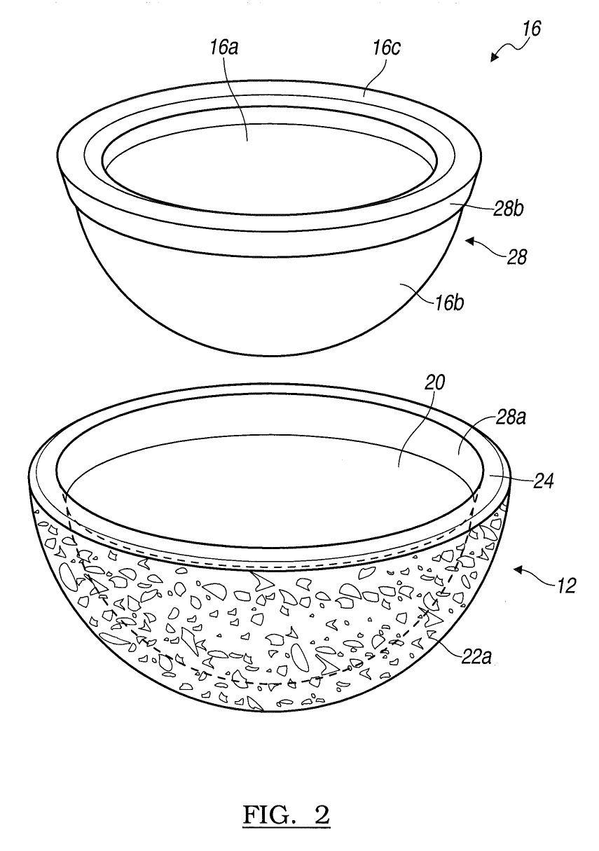

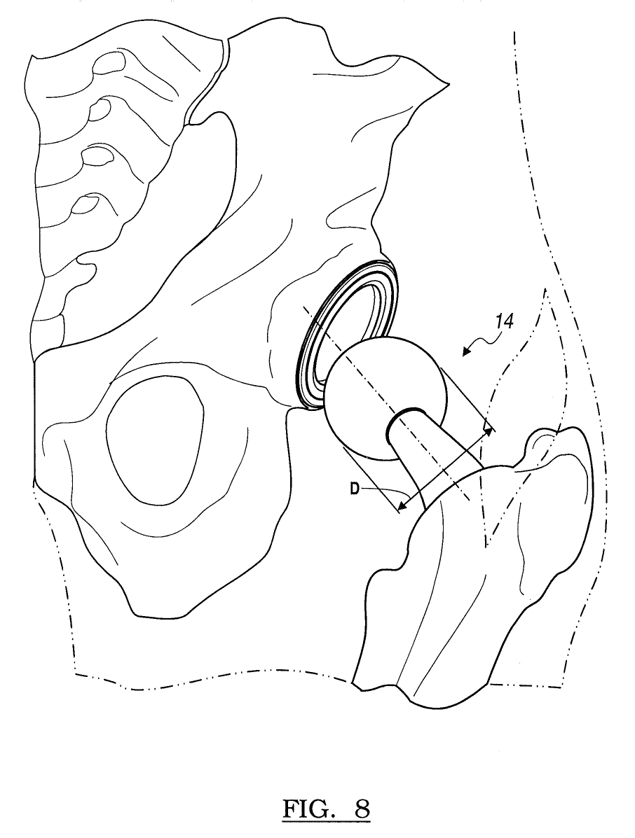

With reference to FIGS. 1 and 2, an exemplary acetabular implant assembly 10 including an exemplary acetabular cup 12 is shown. The acetabular implant assembly 10 can be used to replace an articulating portion of the anatomy, such as a hip joint. In this regard, the acetabular cup 12 can be used to replace an acetabulum, and a bearing liner 16 received within the acetabular cup 12 includes an articulating surface that can cooperate with an articulating member, such as femoral head prosthesis or implant 14 (FIGS. 7 and 8). As will be discussed, acetabular cup 12 can enable the receipt of a larger femoral head implant 14. The use of a larger femoral head implant 14 can reduce the chance of dislocation of the femoral head implant 14 from the acetabular cup 12. In addition, the use of a larger femoral head implant 14 can increase the range of motion of the femoral head implant 14 relative to the acetabular cup 12.

With reference back to FIGS. 1, 7 and 8, the acetabular implant assembly 10 can include the acetabular cup 12, a bearing insert or liner 16 and the femoral head implant 14 (FIGS. 7 and 8). With reference to FIGS. 1-6, the acetabular cup 12 can be generally hemispherical, and can be configured to be received into a prepared acetabulum. The acetabular cup 12 can be comprised of a suitable biocompatible material, and for example, can be composed of a biocompatible polymer.

In one example, the acetabular cup 12 can be composed of a fiber-reinforced polymer, such as a carbon fiber reinforced polyetheretherketone. An exemplary carbon fiber reinforced polyetheretherketone can comprise PEEK-OPTIMA®, which is commercially available from Invibio Ltd. of Lancashire, United Kingdom. The use of a fiber-reinforced polymer for the acetabular cup 12 can enable the formation of a thinner, stronger and stiffer acetabular cup 12.

In this regard, due to the material properties of carbon fiber reinforced polyetheretherketone, the acetabular cup 12 can be formed with a reduced wall thickness T while maintaining sufficient or improved stiffness over a metal or metal alloy acetabular cup.

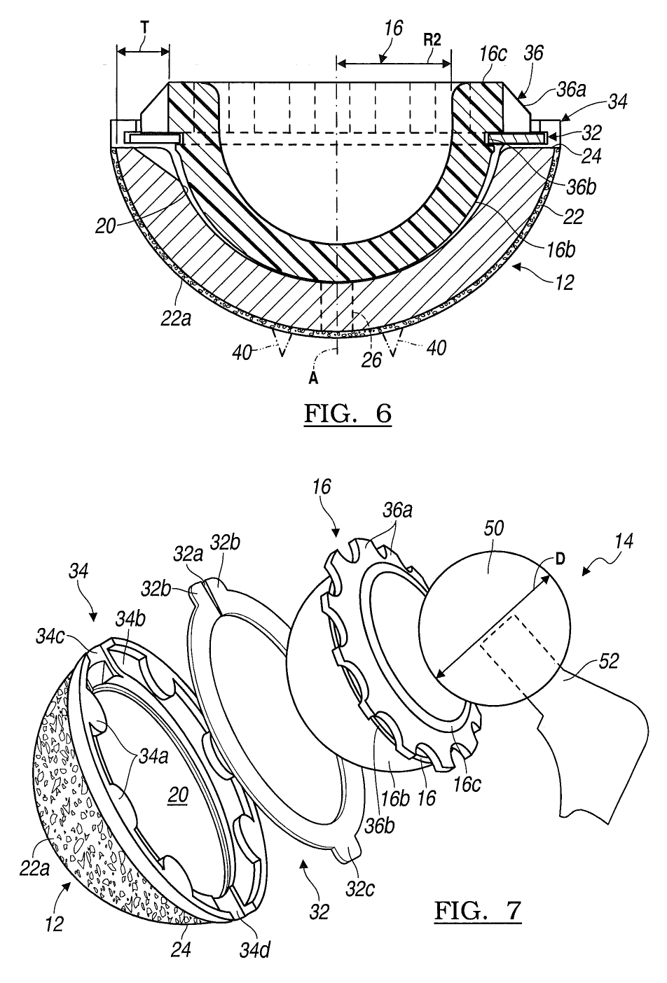

In one example, with reference to FIGS. 3 and 6, the use of a fiber-reinforced polymer for the acetabular cup 12 can enable the acetabular cup 12 to be formed with the wall thickness T, which can have a wall thickness that is about 5 percent to about 50 percent smaller than a metal or metal alloy acetabular cup. The stiffness of the acetabular cup 12 can be at least about 0.01 millimeter (mm) per 2000 Newtona (N), which can be about equal to or greater than a stiffness of a metal or metal alloy acetabular cup having the larger thickness of about 3.0 millimeters (mm) to 7.0 millimeters (mm).

Thus, the use of a fiber-reinforced polymer for the acetabular cup 12, can enable the formation of an acetabular cup 12 having a reduced wall thickness T, without substantially sacrificing the strength or stiffness of the acetabular cup 12. In addition, the use of a fiber-reinforced polymer for the acetabular cup 12 can provide sufficient flexibility for implanting the acetabular cup 12 into the anatomy. In this regard, if the acetabular cup 12 is press-fit into the anatomy, it is desirable for the acetabular cup 12 to have sufficient strength for impaction but also for the acetabular cup 12 to flex to engage the anatomy. The use of the fiber-reinforced polymer can allow the formation of the acetabular cup 12 with reduced wall thickness T while maintaining a stiffness of about 0.1 millimeter (mm) per 2000 Newtons (N) to enable the acetabular cup 12 to be implanted into the anatomy.

With continued reference to FIGS. 3 and 6, the reduction in the wall thickness T of the acetabular cup 12 can result in a larger inner radius R for the acetabular cup 12, which can allow the acetabular cup 12 to accept a femoral head implant 14 having a larger radius or diameter D (FIGS. 7 and 8), as will be discussed further herein.

With continued reference to FIGS. 1-6, the acetabular cup 12 can include an interior bearing engaging surface 20, an exterior bone engaging surface 22 and a rim 24. The acetabular cup 12 can be formed through any suitable polymer processing method, such as through injection molding. The use of injection molding to form the acetabular cup 12 may reduce the manufacturing costs of forming the acetabular cup 12 when compared to the manufacture of a metal or metal alloy acetabular cups that are cast or milled.

The acetabular cup 12 can also include at least one aperture 26, if desired. It should be noted that although the aperture 26 is illustrated herein in phantom in FIGS. 3 and 6 as being formed at an apex A of the acetabular cup 12, the aperture 26 could be formed at any desired location, such as below the rim 24. The aperture 26 can be configured to receive a tool to couple the acetabular cup 12 to the anatomy, such as an impact tool to press-fit the acetabular cup 12 into the anatomy. Thus, if employed, the aperture 26 can be used to enable the insertion and extraction of the acetabular cup 12. It should be understood, however, that the aperture 26 is optional, as various other techniques could be used to couple the acetabular cup 12 to the anatomy, as will be discussed herein.

With reference to FIG. 2, the interior surface 20 of the acetabular cup 12 can be concave and generally smooth. The interior surface 20 can be configured to receive the bearing liner 16, and to couple the bearing liner 16 to the interior surface 20. As the interior surface 20 can be coupled to the bearing liner 16 via any suitable known technique, the coupling of the interior surface 20 to the bearing liner 16 will not be discussed in great detail herein. Briefly, however, in an example of a ceramic, metal or metal alloy bearing liner 16, with reference to FIGS. 1-3, the bearing liner 16 can be coupled to the acetabular cup 12, via a taper lock connection 28.

The taper lock connection 28 can provide a frictional or mechanical lock between the bearing liner 16 and the acetabular cup 12. In this regard, with reference to FIGS. 2 and 3, the taper lock connection 28 can be formed by the frictional engagement of a first taper 28a with a second taper 28b. In this example, the first taper 28a can be formed on the interior surface 20 of the acetabular cup 12, and generally, can be formed adjacent to the rim 24. The first taper 28a can define any appropriate angle such as a conventional locking taper. In one example, with reference to FIG. 3, the taper 28a can be formed at an about 1° to about 25° angle A1 with respect to a vertical axis V1.

With reference to FIGS. 2 and 3, the second taper 28b can be formed on an exterior surface 16a of the bearing liner 16. The second taper 28b can be positioned to frictionally engage the first taper 28a of the acetabular cup 12 when the bearing liner 16 is properly positioned within the acetabular cup 12. The second taper 28b can define any appropriate angle such as a conventional locking taper. In one example, with reference to FIG. 3, the second taper 28b can be formed at an about 1° to about 25° angle A2 with respect to the vertical axis V1. Further detail regarding an exemplary taper lock connection 28 including a first taper and a second taper is disclosed in co-pending U.S. patent application Ser. No. 11/365,895, filed on Feb. 28, 2006, and incorporated herein by reference.

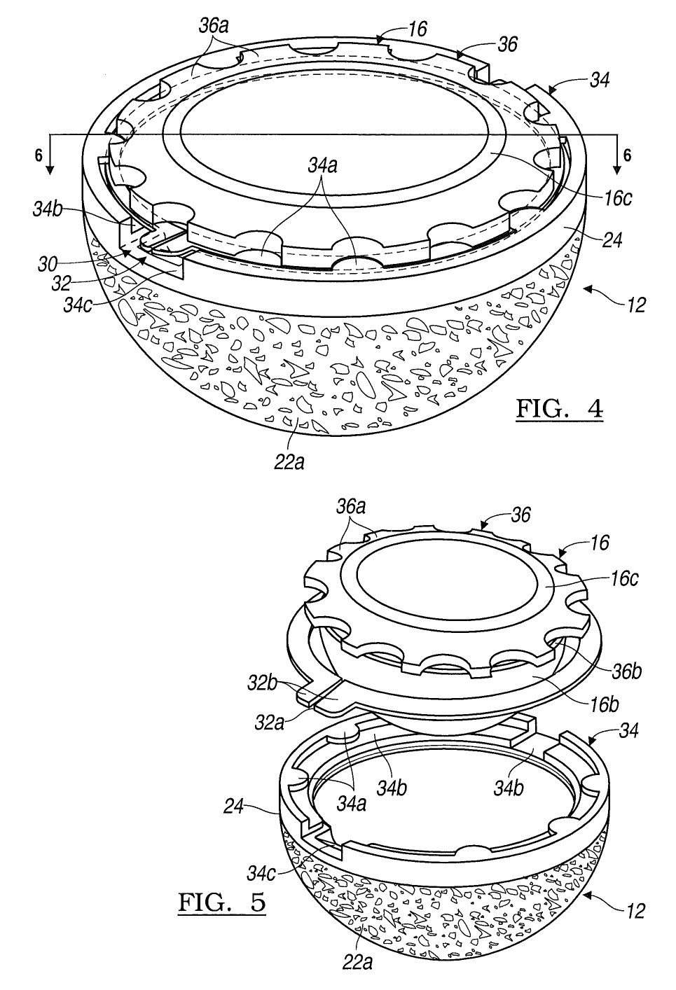

In another example, such as in the case of a polymer bearing liner 16, with reference to FIGS. 4-7, the bearing liner 16 can be coupled to the interior surface 20, via a ring lock connection 30. As a suitable ring lock connection can comprise the RingLoc® system commercially available from Biomet, Inc. of Warsaw, Ind., or a ring lock system disclosed in co-pending U.S. patent application Ser. No. 11/714,991, filed on Mar. 7, 2007, and incorporated herein by reference, the ring lock connection 30 will not be described in great detail herein. Briefly, however, the ring lock connection 30 can include a ring 32, a first interlocking portion 34 and a second interlocking portion 36.

With reference to FIGS. 5-7, the ring 32 can be composed of a biocompatible material, and include a slit 32a, a pair of fingers 32b and a protrusion 32c. The slit 32a can enable the ring 32 to radially expand to couple the bearing liner 16 to the acetabular cup 12. The fingers 32b can be received into a portion of the first interlocking portion 34 when the bearing liner 16 is coupled to the acetabular cup 12, as shown in FIGS. 4 and 5. The protrusion 32c can be diametrically opposite the fingers 32b, and can also be received into a portion of the first interlocking portion 34 (FIGS. 6 and 7).

With reference to FIGS. 4-7, the first interlocking portion 34 can be coupled to the rim 24 of the acetabular cup 12. The first interlocking portion 34 can include one or more lobbed fingers 34a, a groove 34b, a first slot 34c and a second slot 34d. The lobbed fingers 34a can alternate about a circumference of the rim 24, and can engage the second interlocking portion 36, as will be discussed. The groove 34b can be recessed or inboard of the rim 24, and can be sized to receive a portion of the ring 32 when the bearing liner 16 is coupled to the acetabular cup 12. The first slot 34c can be diametrically opposed from the second slot 34d, and can receive the fingers 32b of the ring 32. The second slot 34d can receive the protrusion 32c of the ring 32, when the bearing liner 16 is coupled to the acetabular cup 12.

With continued reference to FIGS. 4-7, the rim 16c of the bearing liner 16 includes a second interlocking portion 36. The second interlocking portion 36 can include one or more lobbed notches 36a and a groove 36b. The lobbed notches 36a can cooperate with the lobbed fingers 34a of the first interlocking portion 34 to prevent the rotation of the bearing liner 16 within the acetabular cup 12 when the bearing liner 16 is coupled to the acetabular cup 12. The groove 36b can be formed on the exterior surface 16b of the bearing liner 16, and generally, can be formed adjacent to the rim 16c of the bearing liner 16. The groove 36b can cooperate with the groove 34b of the first interlocking portion 34 to receive a portion of the ring 32. Thus, once assembled, the ring 32 can nest within the groove 34b of the first interlocking portion 34 and the groove 36b of the second interlocking portion 36 to couple the bearing liner 16 to the acetabular cup 12 (FIG. 6).

With reference to FIGS. 1-6, the exterior surface 22 of the acetabular cup 12 can be configured to mate with a prepared acetabulum. In one example, the exterior surface 22 can be roughened to engage acetabulum. Further, if desired, the exterior surface 22 can be coated with a biocompatible material 22a to facilitate boney in-growth, such as a porous metal matrix, for example a porous plasma spray, calcium phosphate, which can include hydroxyapatite, a biologically active substance, such as a bone morphogenic protein, a growth factor, a peptide, antibiotic, etc. In addition, it should be understood that the exterior surface 22 can be both roughened and then coated prior to coupling the acetabular cup 12 with the anatomy.

Further, if desired, the exterior surface 22 can also include one or more engagement features 40, as illustrated in phantom in FIGS. 3 and 6. The engagement features 40 can further couple the acetabular cup 12 to the anatomy. In one of various examples, the engagement features 40 can comprise spikes, however, the engagement features 40 can comprise any feature suitable for engaging the tissue of the anatomy. For example, the engagement features 40 can comprise fins, projections, posts, etc.

The rim 24 of the acetabular cup 12 can include one or more features to couple the bearing liner 16 to the acetabular cup 12, as discussed previously herein.

With reference to FIGS. 3 and 6, the bearing liner 16 can be received within and coupled to the acetabular cup 12. Due to the decreased wall thickness T of the acetabular cup 12, the bearing liner 16 can have an increased radius R2. The increased radius R2 of the bearing liner 16 can enable the receipt of the femoral head implant 14 having a larger radius or diameter D. The bearing liner 16 can be generally concave and hemispherical. The bearing liner 16 can be composed of a biocompatible material. In one example, the bearing liner 16 can be composed of a biocompatible metal or metal alloy, such as titanium, titanium alloy, stainless steel, cobalt-chromium-molybedenum alloy, etc. In addition, the bearing liner 16 can be composed of a biocompatible ceramic or polymer. The bearing liner 16 can include the exterior surface 16a, the interior surface 16b and the rim 16c.

In one example, the exterior surface 16a can couple the bearing liner 16 to the acetabular cup 12, via the taper lock connection 28, as discussed previously herein with regard to FIGS. 1-3. In another of various examples, the exterior surface 16b and the rim 16c can cooperate to couple the bearing liner 16 to the acetabular cup 12, via a ring lock connection 30, as discussed previously herein with regard to FIGS. 4-7. The interior surface 16b of the bearing liner 16 can be generally smooth and can receive the femoral head implant 14 (FIGS. 7 and 8). Thus, the bearing liner 16 can enable the femoral head implant 14 to articulate relative to the acetabular cup 12.

With reference to FIGS. 7 and 8, the femoral head implant 14 can be received within the bearing liner 16. Generally, the femoral head implant 14 can be composed of a suitable biocompatible material. In one example, the femoral head implant 14 can be composed of a biocompatible metal or metal alloy, such as titanium, titanium alloy, stainless steel, cobalt-chromium-molybedenum alloy. In addition, the femoral head implant 14 can be composed of a biocompatible ceramic. An exemplary femoral head implant 14 can be substantially similar to Biolox Delta, commercially available from CeramTec AG, of Plochingen, Germany, or any suitable femoral head implant 14 commerically available from Biomet, Inc. of Warsaw, Ind., having a larger radius or diameter D. As the femoral head implant 14 can be generally known, the femoral head implant 14 will not be discussed in great detail herein. Briefly, however, the femoral head implant 14 can comprise a head 50 and a stem 52.

With continued reference to FIGS. 7 and 8, the head 50 can include the larger radius or diameter D, which can be sized for articulation within the bearing liner 16. Generally, the radius or diameter D can be about 5 to about 50 percent larger than a radius or diameter of a femoral head implant 14 employed with a metal or metal alloy based acetabular cup 12. The large radius or diameter D of the femoral head implant 14 can enable the femoral head implant 14 to move or articulate relative to the acetabular cup 12 with a greater range of motion, as discussed.

With regard to FIGS. 1-7, in order to assemble the acetabular implant assembly 10, the acetabular cup 12 can be guided into a prepared anatomy. Generally, the anatomy can be prepared by reaming the acetabulum with a reamer, as described in U.S. patent application Ser. No. 11/365,895, previously incorporated by reference herein.

With the anatomy prepared, the acetabular cup 12 can be coupled to the anatomy (FIG. 8). In one example, the acetabular cup 12 can be coupled to the anatomy by press-fitting the acetabular cup 12 to the acetabulum, as also discussed in U.S. patent application Ser. No. 11/365,895, previously incorporated by reference herein. The acetabular cup 12 can be press-fit into the anatomy by the application of a force by an impact tool, for example. As discussed, the roughened and/or coated surface of the acetabular cup 12 can enable boney in-growth to couple the acetabular cup 12 to the anatomy. In addition, if the acetabular cup 12 includes one or more engagement features 40, the press-fitting of the acetabular cup 12 can drive the engagement features 40 into the anatomy to further couple the acetabular cup 12 to the anatomy.

After the acetabular cup 12 is coupled to the anatomy, the bearing liner 16 can be coupled to the interior surface 22 of the acetabular cup 12 via the taper lock connection (FIGS. 1-3), the ring lock connection (FIGS. 4-7), etc. as discussed previously herein. With the bearing liner 16 coupled to the acetabular cup 12, the femoral head implant 14 can be guided into engagement with the acetabular cup 12 (FIG. 8). As the acetabular cup 12 has a reduced wall thickness T, the bearing liner 16 can have the increased radius R2, and thus, the femoral head implant 14 can have the increased radius or diameter D. The ability to use the femoral head implant 14 with an increased radius or diameter D can increase the range of motion of the femoral head implant 14 relative to the acetabular cup 12, and can also reduce the dislocation of the acetabular cup 12 from the femoral head implant 14.

In addition, since both the bearing liner 16 and the femoral head implant 14 can be composed of a metal or metal alloy material, the wear resulting from the contact between the bearing liner 16 and the femoral head implant 14 can be reduced as compared to the wear associated with using two different materials. Further, the use of a polymeric material to form the acetabular cup 12 can reduce manufacturing costs associated with the manufacture of the acetabular cup 12, by allowing the acetabular cup 12 to be formed by injection molding, for example.

While specific examples have been described in the specification and illustrated in the drawings, it will be understood by those of ordinary skill in the art that various changes may be made and equivalents may be substituted for elements thereof without departing from the scope of the present disclosure. Furthermore, the mixing and matching of features, elements and/or functions between various examples is expressly contemplated herein so that one of ordinary skill in the art would appreciate from this disclosure that features, elements and/or functions of one example may be incorporated into another example as appropriate, unless described otherwise, above. Moreover, many modifications may be made to adapt a particular situation or material to the teachings of the present disclosure without departing from the essential scope thereof. Therefore, it is intended that the present disclosure not be limited to the particular examples illustrated by the drawings and described in the specification as the best mode presently contemplated for carrying out this disclosure, but that the scope of the present disclosure will include any embodiments falling within the foregoing description.

What is claimed is:

1. An acetabular implant assembly for replacing an articulating portion of an anatomy comprising:
a fiber-reinforced polymeric cup having an interior surface and an exterior surface that engages the anatomy;
a metal or ceramic bearing liner coupled to the interior surface of the cup via a taper lock connection, the metal or ceramic bearing liner having an interior bearing surface; and
an articulating member movable within the interior bearing surface to replace the articulating portion of the anatomy.

2. The acetabular implant assembly of claim 1, wherein the cup is an acetabular cup, and the articulating member is a femoral head implant.

3. The acetabular implant assembly of claim 1, wherein the cup is reinforced with carbon fibers.

4. The acetabular implant assembly of claim 2, wherein the acetabular cup is composed of a polyetheretherketone reinforced with carbon fibers.

5. The acetabular implant assembly of claim 4, wherein the acetabular cup has a wall thickness, which is between about 1.0 millimeter (mm) to about 5.0 millimeters (mm).

6. The acetabular implant assembly of claim 5, wherein the femoral head implant includes a ball coupled to a stem, and the wall thickness of the acetabular cup enables the ball to have a diameter that is between about 22 millimeters (mm) to about 60 millimeters (mm).

7. The acetabular implant assembly of claim 1, wherein the metal bearing liner and the articulating member are each formed from a biocompatible material selected from the group comprising: titanium, titanium alloy, ceramic, stainless steel, cobalt-chromium-molybedenum alloy and combinations thereof.

8. The acetabular implant assembly of claim 2, wherein the femoral head implant is formed from a biocompatible metal or metal alloy.

9. An acetabular implant assembly for replacing an articulating portion of an anatomy comprising:
an acetabular cup composed of a carbon fiber-reinforced polyetheretherketone material and having an exterior surface that engages the anatomy and an interior surface;
a metal bearing liner coupled to the interior surface of the acetabular cup via a taper lock connection, the bearing liner including an articulated bearing surface; and
a metal femoral head implant including a head that articulates within the interior surface of the bearing liner.

10. The acetabular implant assembly of claim 9, wherein the acetabular cup has a wall thickness, which is between about 1.0 millimeter (mm) to about 5.0 millimeters (mm).

11. The acetabular implant assembly of claim 10, wherein the femoral head implant includes a ball coupled to a stem, and the wall thickness of the acetabular cup enables the ball to have a diameter that is between about 22 millimeters (mm) to about 60 millimeters (mm).

12. The acetabular implant assembly of claim 10, wherein the metal bearing liner and the metal femoral head implant are each formed from a biocompatible material selected from the group comprising: titanium, titanium alloy, stainless steel, cobalt-chromium-molybedenum alloy and combinations thereof.

13. A method of replacing an articulating portion of an anatomy with an acetabular implant assembly comprising:
inserting a fiber-reinforced polymeric acetabular cup into a prepared portion of the anatomy;
coupling a metal bearing liner to an interior surface of the polymeric acetabular cup; and
coupling a metal femoral head implant to the metal bearing liner.

14. The method of claim 13, wherein inserting the polymeric acetabular cup into a prepared portion of the anatomy further comprises:
inserting a carbon-fiber reinforced polyetheretherketone acetabular cup into the anatomy.

15. The method of claim 13, wherein coupling the metal femoral head implant to the metal bearing liner further comprises:
articulating the metal femoral head implant within the metal bearing liner.

16. The method of claim 13, wherein coupling the metal bearing liner to the interior surface of the polymeric acetabular cup further comprises:
coupling the metal bearing liner to the polymeric acetabular cup with a taper lock connection.
